(19) United States
(12) Patent Application Publication
(10) Pub. No.: US 2001/0021804 A1
(43) Pub. Date: Sep. 13, 2001

(54) METHOD AND APPARATUS FOR PERFORMING MULTI-CHROMATIC IMAGING SPECTROPHOTOMETRY USING A SINGLE DETECTOR

(76) Inventor: RICHARD G. NADEAU, NORTH EAST, MD (US)

Correspondence Address: STERNE KESSLER GOLDSTEIN & FOX, 1100 NEW YORK AVENUE N.W., SUITE 600, WASHINGTON, DC, 200053934

(21) Appl. No.: 09/227,812

(22) Filed: Jan. 11, 1999

(51) Int. Cl. A61B 5/00

(52) U.S. Cl. 600/407; 600/310; 600/476

(57) ABSTRACT

A system and method for multi-chromatic imaging that requires a single detector are described. A light source sequentially produces multiple light wavelengths for illumination of a subject. An optical filter filters the light wavelengths, and directs them to the subject, and then filters the transmitted or reflected light, and directs it to an image detector. The image detector sequentially receives each transmitted or reflected light wavelength with a different frame or field, and then produces an detected image signal for an analyzer. A video sync separator separates the composite video image signal from the image detector into the signal components necessary for sequencing the light source. A sequence driver uses these separated signal components to create sequencing signals for driving the light source. These sequencing signals keep the wavelengths produced by the light source synchronized with the respective frames or fields of the image detector

### CROSS-REFERENCE TO RELATED APPLICATIONS

This application is related to commonly owned, co-pending U.S. patent application Ser. No. 08/860,363, filed Jun. 5, 1997, entitled “Method and Apparatus for Reflected Imaging Analysis,” which is incorporated by reference. This application is also related to commonly owned, co-pending U.S. patent application Ser. No. [to be assigned], filed Nov. 5, 1998, (Attorney Docket No. 1637.0150000), entitled “Method and Apparatus for Providing High Contrast Imaging,” which is also incorporated by reference.

### BACKGROUND OF THE INVENTION

1. Field of the Invention

The present invention generally relates to transmitted and reflected light analysis. More particularly, the present invention relates to the use of multi-chromatic imaging spectrophotometry to perform non-invasive analysis of a subject's vascular system with a single detector.

2. Related Art

Spectrophotometry involves analysis based on the absorption or attenuation of electromagnetic radiation by matter at one or more wavelengths of light. The instruments used in this analysis are referred to as spectrophotometers. A simple spectrophotometer includes: a source of radiation, such as, for example, a light bulb; a means of spectral selection such as a monochromator containing a prism or grating or colored filter; and one or more detectors, such as, for example, photocells, which measure the amount of light transmitted and/or reflected by the sample in the selected spectral region.

In opaque samples, such as solids or highly absorbing solutions, the radiation reflected from the surface of the sample may be measured and compared with the radiation reflected from a non-absorbing or white sample. If this reflectance intensity is plotted as a function of wavelength, it gives a reflectance spectrum. Reflectance spectra are commonly used in matching colors of dyed fabrics or painted surfaces. Additionally, U.S. patent application Ser. No. 08/860,363 to Groner et al., “Method and Apparatus for Reflected Imaging Analysis” (referred to hereinafter as the '363 application), describes the use of reflectance spectra to image the vascular system of a subject.

Transmission spectrophotometry involves measuring the radiation transmitted through a sample. It is conventionally useful for quantitative analysis because Beer's law (inversely relating the logarithm of measured intensity linearly to concentration) can be used.

Generally, spectrophotometry involves the measurement of reflected or transmitted light spectrum intensity. Classical spectrophotometry involves comparing the intensity of measured light at a single point in space, at different times. Imaging spectrophotometry involves comparing the intensity of measured light at two or more different points in an image, at the same time.

Many existing spectrophotometric devices for performing transmitted and reflected imaging utilize monochromatic imaging means. In a monochromatic imaging system, typically a single light source wavelength illuminates a subject, and a single imaging detector records the transmitted or reflected image in sequential frames. The benefits of monochromatic devices include their low cost and ease of calibration.

Existing spectrophotometric devices, such as described by the above-referenced '363 application, utilize multi-chromatic means for producing images. Such devices typically require at least two imaging detectors to measure the amount of light transmitted and/or reflected by the sample in the selected spectral region. Bi-chromatic devices typically employ a first detector to measure light at the target wavelength, and employ a second detector to measure light at a reference wavelength. A light source is configured to produce the two different wavelengths of light.

Bi-chromatic imaging systems apply a correction function to a raw reflected image to produce a corrected reflected image. For a bi-chromatic correction, two wavelengths of light, λ₁ and λ₂, are selected. A reflected image is captured while illuminating the subject with each wavelength. By subtracting the intensity of the λ₂ image from the intensity of the λ₁ image, all parameters that affect both λ₁ and λ₂ in the same manner are canceled out, and are thus eliminated, in the resulting (λ₁−λ₂) image. For example, when visualizing a patient's vascular system, such parameters that may be eliminated include effects of blood cell scattering, variations of light intensity, depth of penetration, angle of light, and pigmentation of tissue covering the imaged portion of the subject. The resulting (λ₁−λ₂) image incorporates the effect of only those parameters that affect λ₁ and λ₂ differently. If the subject is in motion, the λ₁ image and λ₂ image must be detected close in time for effective imaging. Monochromatic systems are not capable of correcting for such undesirable image parameters. Bi-chromatic imaging systems eliminate these undesirable parameters, and hence can provide higher accuracy spectrophotometry than monochromatic imaging systems. Refer to the above-referenced '363 application for more background on bi-chromatic imaging systems.

For instance, bi-chromatic imaging could be used to analyze hemoglobin concentration. A wavelength of 550 nm is located near the center of an absorption band for hemoglobin. A wavelength of 650 nm is a non-absorbing wavelength for hemoglobin. These wavelengths are useful for bi-chromatic imaging because they are sufficiently close, so that undesirable parameters have the same affect on both. They also have a sufficient spectral spread, so that sufficient signal difference (λ₁−λ₂) is provided. With such a spectral spread, the difference in intensity of reflected light is a function of the concentration of hemoglobin.

The multi-detector approach suffers from certain limitations. For example, the detectors most effectively used for high resolution imaging applications are CCD cameras. A bi-chromatic approach requires two CCD cameras, and requires an image separator to direct the reflected light to each respective camera. It is expensive to have to purchase a CCD camera for each wavelength of light detected. It is desirable to have a multi-chromatic reflected imaging system that has the cost benefits of single detector monochromatic systems with the improved accuracy of the multi-detector multi-chromatic approach.

Additionally, using two or more CCD cameras creates problems of alignment or registration of the two detectors. Problems with intensity calibration across both detectors can also occur.

The limitations of the multi-detector approach also apply equally to transmitted imaging analysis. Transmitted imaging is typically used with in vitro analysis. With transmitted imaging, the light source would form a light path through the subject, directly to the image separator, which would separate the image and send the respective image portions to multiple image detectors. Again, multiple CCD cameras are required, with the consequent cost problem and other described limitations. It would be desirable to have a multi-chromatic transmitted imaging system with the benefits of monochromatic systems and the improved accuracy of the multi-chromatic approach.

### SUMMARY OF THE INVENTION

The present invention is directed to a method and apparatus for performing bi-chromatic reflected imaging spectrophotometry using a single detector with interline capability. In one aspect, an illuminating region illuminates a subject alternately with a first wavelength of light and a second wavelength of light from a light source. The illumination of the subject by the first and second wavelengths of light is synchronized with the alternate fields or frames of the single detector. The single detector signals used to integrate the alternate fields or frames of the single detector are provided as synchronization signals to the light source. A detecting region is used to detect the first wavelength and second wavelength of light reflected from the subject. The wavelengths are respectively detected by alternate fields or frames of the single detector. The detected light is used to perform bi-chromatic imaging spectrophotometry.

In a further aspect of the present invention, a method and apparatus for performing multi-chromatic reflected imaging spectrophotometry using a single detector with multiple frame or field capability is provided. An illuminating region illuminates the subject sequentially with a plurality of light wavelengths from a light source. The illumination of the subject by the first and second wavelengths of light is synchronized with the sequential fields or frames of the single detector. The single detector signals used to integrate the sequential frames of the single detector are provided as synchronization signals to the light source. A detecting region detects the plurality of light wavelengths reflected from the subject respectively with sequential frames of the single detector. This detected light is used to perform multi-chromatic imaging spectrophotometry.

In a further aspect of the present invention, a method and apparatus for performing bi-chromatic transmitted imaging spectrophotometry using a single detector with interline capability is provided. An illuminating region illuminates a subject alternately with a first wavelength of light and a second wavelength of light from a light source. The illumination of the subject by the first and second wavelengths of light is synchronized with the alternate fields or frames of the single detector. The single detector signals used to integrate the alternate fields or frames of the single detector are provided as synchronization signals to the light source. A detecting region detects the first wavelength and second wavelength of light transmitted through the subject respectively with alternate fields or frames of the single detector. The detected light is used to perform bi-chromatic imaging spectrophotometry.

In still a further aspect of the present invention, a method and apparatus for performing multi-chromatic transmitted imaging spectrophotometry using a single detector with multiple field or frame capability is provided. An illuminating region illuminates a subject sequentially with a plurality of light wavelengths from a light source. The illumination of the subject by the first and second wavelengths of light is synchronized with the sequential fields or frames of the single detector. The single detector signals used to integrate the sequential fields or frames of the single detector are provided as synchronization signals to the light source. A detecting region detects the plurality of light wavelengths transmitted through the subject respectively with sequential fields or frames of the single detector. The detected light is used to perform multi-chromatic imaging spectrophotometry.

Features and Advantages

It is a feature of the present invention that it provides for multi-chromatic imaging and requires only a single image detector, does not require an image separator, and does not require a spectral selector for each image detector. It advantageously provides a multi-chromatic reflected imaging system that has the cost benefits of monochromatic systems with the improved accuracy of the multi-chromatic approach.

A further advantage of the present invention is that it does not have the problems of alignment or registration of multiple detector environments. It also advantageously eliminates the problem with intensity calibration across multiple detectors.

A still further advantage of the present invention is that it provides for precise synchronization between light wavelength and image detector field or frame by using the image detector's own frame integration signals for synchronization.

A still further advantage of the present invention is that it provides all these benefits in both a reflected imaging environment and transmitted imaging environment.

### BRIEF DESCRIPTION OF THE FIGURES

Further features and advantages of the invention, as well as the structure and operation of various embodiments of the invention, are described in detail below with reference to the accompanying drawings. In the drawings, like reference numbers generally indicate identical, functionally similar, and/or structurally similar elements. The drawing in which an element first appears is indicated by the left-most digit(s) in the corresponding reference number.

FIG. 1 is a block diagram of a conventional bi-chromatic reflected imaging environment for non-invasive in vivo spectrophotometric analysis, with dual image detectors;

FIG. 2 is a block diagram of a bi-chromatic reflected imaging environment for non-invasive in vivo spectrophotometric analysis, with a single image detector, according to the present invention;

FIG. 3 is a block diagram of a sequence driver according to the present invention;

FIG. 4 is a block diagram of a single detector bi-chromatic imaging apparatus according to a preferred embodiment of the present invention;

FIG. 5 is a circuit diagram of a sequence drive circuit according to a preferred embodiment of the present invention; and

FIG. 6 is a comparison of two signal waveforms, showing the relationship between the vertical sync signal and horizontal sync signal.

### DETAILED DESCRIPTION OF THE PREFERRED EMBODIMENTS

The present invention is directed to a method and apparatus for performing multi-chromatic imaging spectrophotometry using a single detector. Generally speaking, the invention is useful for performing multi-chromatic imaging, without having to use multiple detectors.

The present invention uses a single image detector, which has multiple frames or fields. For instance, the image detector may be an interline CCD camera. A CCD image frame typically consists of two image fields, an even field and an odd field, which are interlaced to make the composite frame. The multiple frames or fields sequentially detect the corresponding wavelengths of light transmitted through or reflected from a subject by a light source. The image detector provides signals which control the timing of the light source. These signals are used internally by the image detector to control the integration of the multiple image detector frames or fields. By providing these signals to the light source, precise synchronization between light wavelengths and image detector frames or fields is effected. Each of the wavelengths of light produced by the light source are synchronized by the image detector control signals with a corresponding frame or field of the image detector. In this manner, only a single detector is required to detect the transmitted or reflected image for all wavelengths of the light source. In a preferred embodiment, where bi-chromatic imaging is implemented using a single interline CCD camera, the synchronization signals which gate the integration of the separate frames, or fields, of the camera are used to switch the light source, such that one wavelength is integrated on the “odd field” and another wavelength is integrated on the “even field.”

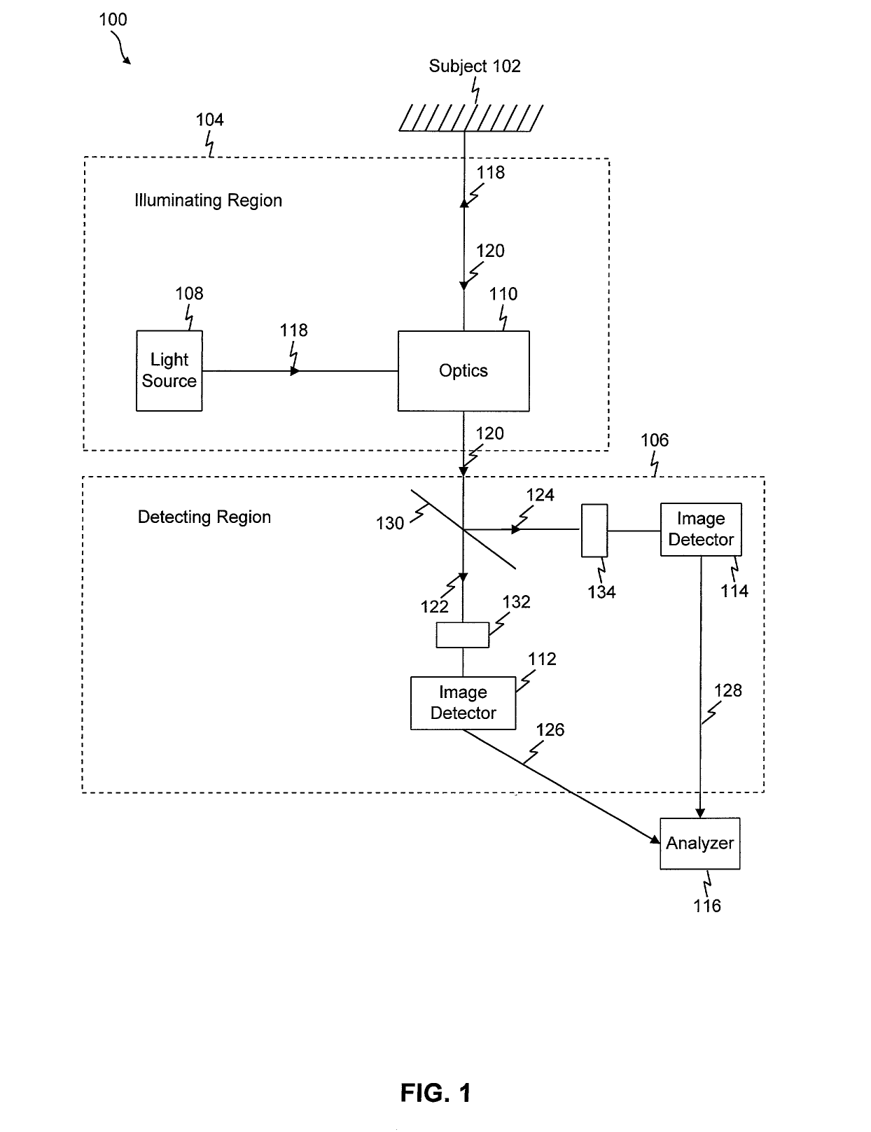

A previously developed system using two detectors was described in the '363 application. For convenience, a simplified diagram of that system is shown in FIG. 1, and is described below. In a preferred embodiment, the present invention is implemented as a bi-chromatic reflected imaging system for spectrophotometric analysis, using the separate fields of a single interline CCD camera to detect the two source wavelengths. For illustrative purposes, the invention is described herein with reference to this preferred embodiment. It should be understood, however, that the invention is not limited to this embodiment. The invention can be used in any application involving multi-chromatic imaging using a single detector.

Previously Developed System

FIG. 1 illustrates a previously developed bi-chromatic reflected imaging environment 100 for non-invasive in vivo spectrophotometric analysis, using dual image detectors. Environment 100 includes a light source 108 for illuminating tissue of a subject (shown generally at 102). An optics 110 is used to form a light path 118 between light source 108 and the illuminated subject 102 and a reflected light path 120. The image reflected from subject 102 travels along reflected light path 120. An image separator 130 is placed in reflected light path 120 between optics 110 and image detectors 112 and 114, for separating the reflected image into a first portion 122 and a second portion 124. First portion 122 of the reflected image passes through first filter 132, and is detected by image detector 112. Second portion 124 passes through second filter 134, and is detected by image detector 114. Image detector 114 can be the same as or different from image detector 112. Image detector 112 and image detector 114 input detected image signal 126 and detected image signal 128, respectively, to an analyzer 116 for carrying out image correction and analysis.

Light source 108 is a multi-chromatic light source that illuminates subject 102 with at least two light wavelengths, so that multi-chromatic imaging spectrophotometric analysis can take place. Although one light source is shown in environment 100, more than one light source is sometimes used in conventional environments. For example, light source 108 could be two or more light emitting diodes (LEDs). Creating multiple wavelengths from a single light source is well known.

Optics 110 directs the light emitted by light source 108 along light path 118 to the subject 102. Optics 110 also directs the light reflected by subject 102 along reflected light path 120 to image separator 130. Optics 110 includes a beam splitter to form light path 118 between light source 108 and the illuminated subject 102. Conventional optics 110 may also include an objective lens placed co-axially in light path 118 and reflected light path 120, to magnify the reflected image. Image detector 112 and image detector 114 are disposed in the magnified image plane of optics 110.

Additionally, conventional optics 110 may include one or more polarizers. Light from light source 108 may be polarized in a first direction by a first polarizer disposed in light path 118. Thus, the light reflected from subject 102 would be polarized. The Rayleigh scattering component of the light reflected from subject 102, however, is de-polarized. The Rayleigh scattering component is light that is scattered by particles that are small compared to the wavelength of the light. A second polarizer in optics 110 may be oriented at 90° to the first polarizer, and placed in reflected light path 120. Polarizers such as these, having planes of polarization oriented 90° relative to each other are known as “cross-polarizers.” When the reflected light passes through the second polarizer, only the Rayleigh scattering component that has been de-polarized will be transmitted. Rayleigh scattering provides a virtual backlighting effect that significantly increases the contrast and visualization of reflected images, and the ability to perform quantitative analyses using reflected images. Therefore, the use of cross-polarizers in optics 110 is desirable. Refer to the above-referenced '363 application for more background on the use of cross-polarizers.

As shown in FIG. 1, image separator 130 separates the image into two components, which are directed through filters to respective image detectors. In conventional environments, image separator 130 could separate the reflected image into a plurality of portions. Additional image detectors would be used to detect further image portions separated by image separator 130. Conventional image separators include dichroic mirrors or other type of dichroic separators that transmit all light less than a particular wavelength, and reflect all light greater than the particular wavelength. Other suitable image separating means are also conventionally used.

First filter 132 is placed in reflected light path 122 between optics 110 and image detector 112. Second filter 134 is placed in reflected light path 124 between optics 110 and image detector 114. First filter 132 and second filter 134 are used for spectral selection. First filter 132 and second filter 134 can be monochromators, spectral filters, prisms, or gratings. The center values for filters 132 and 134 can be chosen based upon the type of analysis to be conducted. For example, if bilirubin concentration is to be determined, then one of filters 132 or 134 is centered at 450 nm, and the other of filters 132 or 134 is centered at 600 nm.

Image detector 112 and image detector 114 input detected image signal 126 and detected image signal 128 to analyzer 116. Analyzer 116 is typically a computer or other type of processing system suitable for correcting and analyzing the received signals, and performing spectrophotometric analysis.

Structure and Operation of the Present Invention

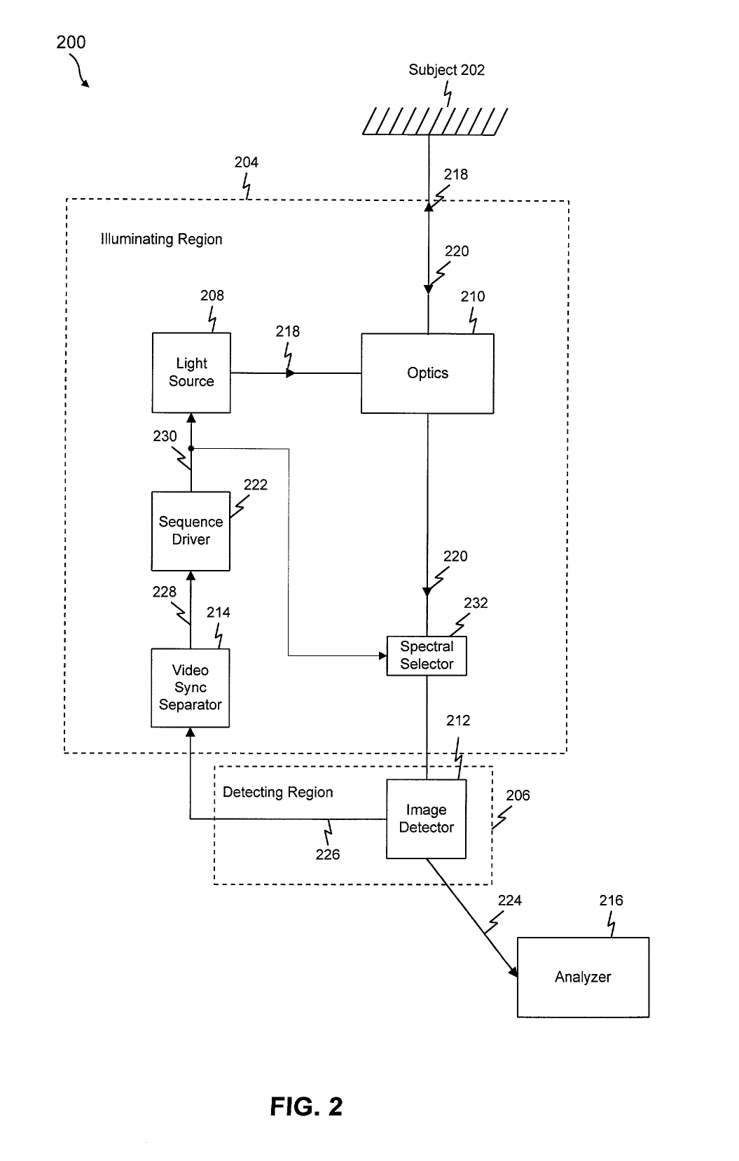

Previously developed imaging environment 100 demonstrated a system that spatially multiplexed multiple wavelengths onto multiple detectors. FIG. 2 shows a block diagram illustrating one embodiment of a single detector environment 200 according to a preferred embodiment of the present invention. Single detector environment 200 demonstrates a system that temporally multiplexes multiple wavelengths onto a single detector. The single detector environment 200 includes an illuminating region 204 for illuminating tissue of a subject (shown generally at 202) and a detecting region 206. The detecting region 206 provides output to an analyzer 216.

Generally, illuminating region 204 reflects light off of subject 202 and directs this reflected light to detecting region 206, which detects the reflected light and provides a detected image signal 224 to analyzer 216. The detecting region 206, in turn, controls the light source located in illuminating region 204, such that the distinct multiple wavelengths of the multi-chromatic light source of illuminating region 204 are synchronized with the multiple detection frames or fields in detecting region 206. Detecting region 206 provides detected image information regarding the reflected wavelengths to the corresponding multiple fields of analyzer 216.

In the current embodiment, illuminating region 204 includes a light source 208, an optics 210, a video sync separator 214, a sequence driver 222, and a spectral selector 232. Spectral selector 232 may be located in either a reflected light path 220 as shown, or in an illuminating light path 218 (not shown). Detecting region 206 includes an image detector 212.

Light source 208 produces a light path 218 used to illuminate subject 202. Although one light source block is shown, it is to be understood that the present invention is not limited to the use of one light source, and more than one light source can be used. In an embodiment where more than one light source is used, each light source can be monochromatic or polychromatic. Light source 208 can be a light capable of being pulsed, a non-pulsed light source providing continuous light, or one capable of either type of operation. Light source 208 can include, for example, a pulsed xenon arc light, a mercury arc light, a halogen light, a tungsten light, a laser, a laser diode, or an LED. Light source 208 can be a source for coherent light, or a source for incoherent light. Light source 208 produces at least two wavelengths of light, depending on the number of detection frames or fields available and numbers of wavelengths of light needed. This light follows light path 218 to optics 210.

Optics 210 is used to form a light path 218 between light source 208 and the illuminated subject 202, and to form a reflected light path 220. The reflected image travels along reflected light path 220 to image detector 212 for detecting the reflected image. Optics 210 may include a beam splitter used to form light path 218 and reflected light path 220.

Image detector 212 has multiple frames or fields for capturing the respective wavelengths of light source 208. The signals used internally by image detector 212 to integrate the multiple frames or fields are used to create synchronization signals for light source 208. Light source 208 uses these synchronization signals to synchronize the multiple wavelengths of light with respective frames or fields of image detector 212. Suitable examples of image detector 212 include, but are not limited to, a camera, a film medium, a CCD camera, or a CMOS camera. For example, video cameras and CCD cameras having a 1,024×512 pixel resolution and 300 Hz framing rate can be used.

Image detector 212 outputs a composite video signal 226 into video sync separator 214. Video sync separator 214 separates the composite video signal 226 into its basic components, including a vertical sync signal and horizontal sync signal. These signals are output from video sync separator 214 as separated video signal 228. In an alternative embodiment, image detector 212 internally separates composite video into its basic components, such as the vertical and horizontal sync signals, and makes these available directly. Hence, in this alternative embodiment, video sync separator 214 is not necessary, and image detector 212 interfaces directly with sequence driver 222.

Sequence driver 222 receives separated video signal 228. Sequence driver 222 uses separated video signal 228 to create a trigger signal 230. Trigger signal 230 is used to trigger the illuminating light wavelengths, such that each wavelength is synchronized with the corresponding field or frame of image detector 212.

In a preferred embodiment, trigger signal 230 is used to trigger light source 208. In this embodiment, light source 208 includes two or more individual light sources, each emitting a different wavelength of light. Spectral selector 232 is not required in this embodiment. Trigger signal 230 triggers light source 208, such that each light source, with its respective light wavelength, illuminates in synchronization with the corresponding field or frame of image detector 212. For example, if light source 208 includes a red light source, a green light source, and a blue light source, sequence driver 222 will create trigger signal 230 such that the red light source is synchronized with the first frame or field of image detector 212, the green light source is synchronized with the second frame or field of image detector 212, and the blue light source will be synchronized with the third frame or field of image detector 212.

In an alternative preferred embodiment, trigger signal 230 is used to trigger spectral selector 232. In this embodiment, light source 208 is preferably a broadband source, and does not require coupling with trigger signal 230. Spectral selector 232 filters light source 208, passing wavelengths of light that are synchronized with corresponding frames or fields of image detector 212. Spectral selector 232 can be, for example, a spinning chopper wheel, a liquid crystal switching bandpass filter, or an acousto-optic tunable filter. The center values for spectral selector 232 can be chosen based upon the type of analysis to be conducted. For example, if hemoglobin concentration is to be determined, then one wavelength of spectral selector 232 is preferably centered at 550 nm and the other wavelength is preferably centered at 650 nm. Spectral selector 232 receives trigger signal 230, which triggers spectral selector 232 such that each wavelength passed by spectral selector 232 is synchronized with a corresponding field or frame of image detector 212.

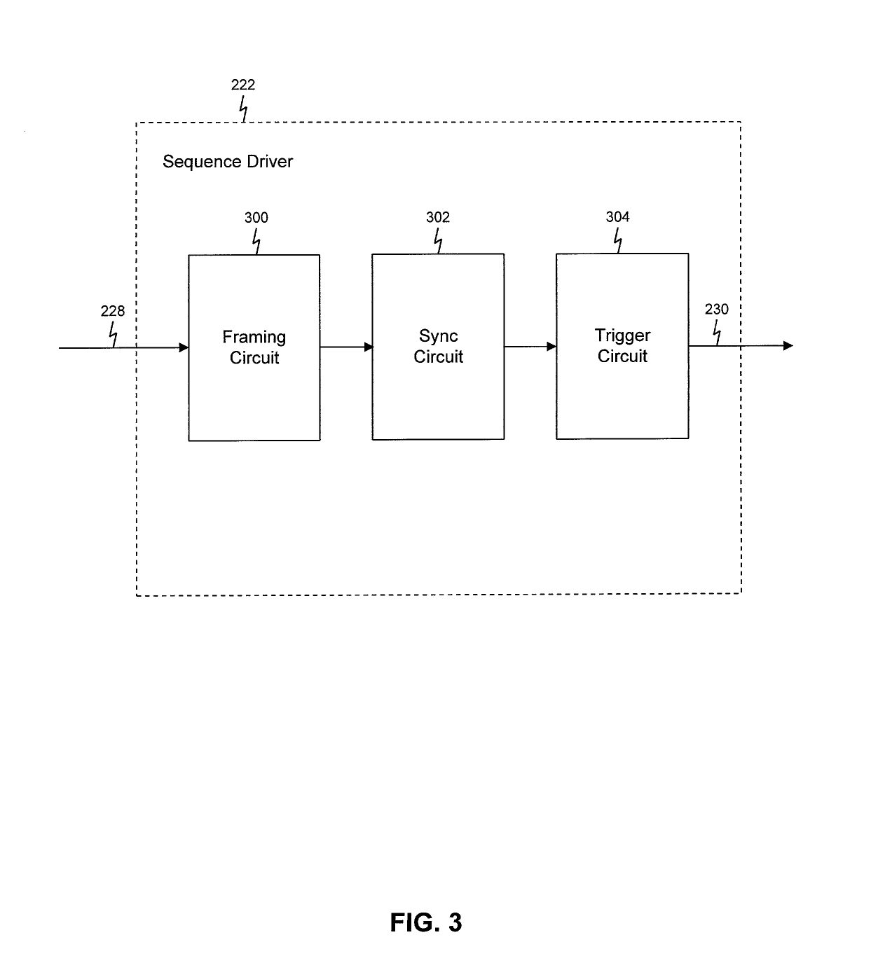

FIG. 3 shows a block diagram of a sequence driver 222. Sequence driver 222 includes a framing circuit 300, a sync circuit 302, and a trigger circuit 304. Sequence driver 222 controls light source 208 or spectral selector 232 such that they produce light wavelengths in synchronization with image detector 212. The framing circuit 300 synchronizes the light wavelengths with the start of the first frame or field of image detector 212. Synchronization circuit 302 synchronizes the illumination of the multiple wavelengths of light with the multiple frames or fields. Trigger circuit 304 triggers each light wavelength Framing circuit 300, sync circuit 302, and trigger circuit 304 may be implemented on a single printed circuit board. Other combinations and materials for implementing sequence driver 222, including an implementation in software, will be apparent to a person skilled in the art and are within the scope of the present invention.

While the block diagram of FIG. 2 describes a reflected imaging environment, it is emphasized that the present invention can also be implemented in a transmitted imaging environment. It will be apparent to a person skilled in the art how to adapt FIG. 2 to a transmitted imaging environment. For example, in a transmitted imaging environment, optics 210 would not require a beam splitter, and subject 202 would be situated in a light path between light source 208 and image detector 212.

Preferred Embodiment

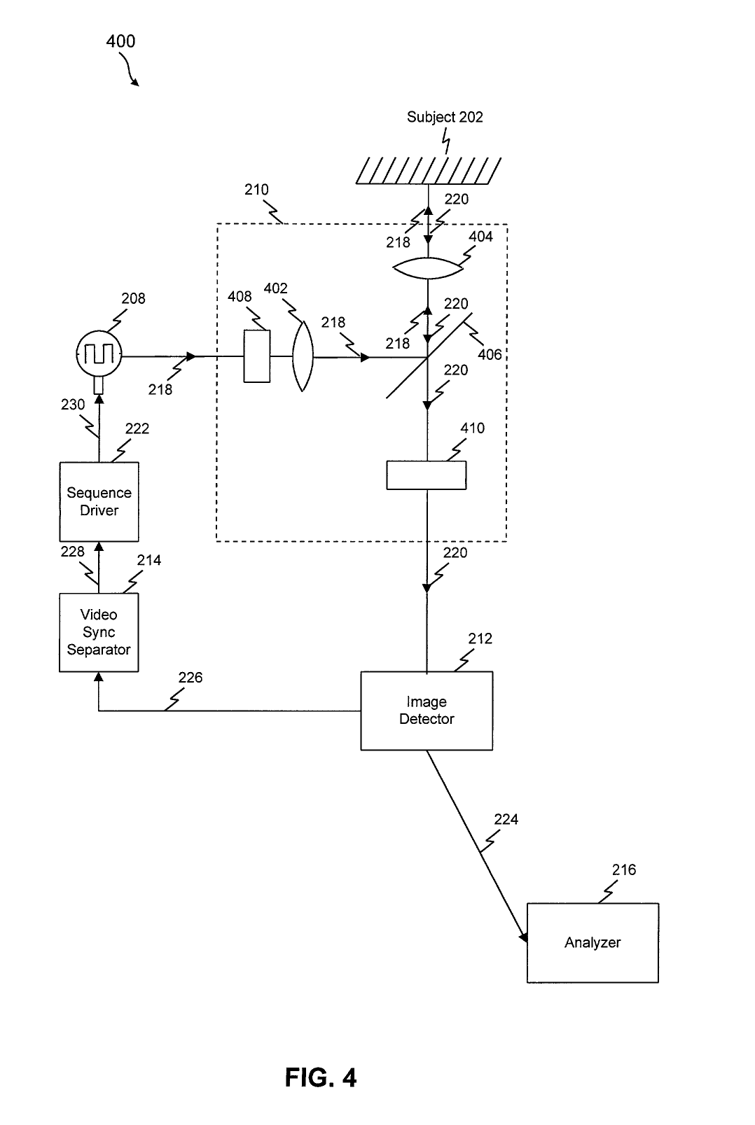

FIG. 4 illustrates a single detector bi-chromatic reflected imaging apparatus 400 used for spectrophotometric analysis according to a preferred embodiment of the present invention. Imaging apparatus 400 is preferably used for non-invasive in vivo analysis of a subject's vascular system. Imaging apparatus 400 includes a light source 208, an optics 210, an image detector 212, a video sync separator 214, and a sequence driver 222. Optics 210 filters and directs light source 208 to the subject 202, illuminating the subject's tissue. Image detector 212 provides reflected image output to analyzer 216. Optics 210 includes a collimating lens 402, an objective lens 404, a beam splitter 406, a first polarizer 408, and a second polarizer 410.

The single detector bi-chromatic imaging apparatus 400 includes a light source 208 for illuminating tissue of a subject 202. In a preferred embodiment, light source 208 includes two LEDs, producing two wavelengths. Light source 208 produces the two wavelengths of light which travel in light path 218 through collimating lens 402. Light source 208 may also include a heat rejection filter. A heat rejection filter is used to reject heat emitted by light source 208.

Optics 210 may include a first polarizer 408, placed between light source 208 and subject 202. First polarizer 408 polarizes light from light source 208. A second polarizer 410 can be placed in reflected light path 220. These first and second polarizers preferably have planes of polarization oriented 90° relative to each other. Polarizers having planes of polarization oriented 90° relative to each other, called crossed-polarizers, are used to eliminate reflected light as described above. Suitable cross-polarizers are available as sheet polarizers from Polaroid Corp., Massachusetts.

In another preferred embodiment of the present invention, light source 208 is itself a source of polarized light, a laser or a laser diode, so that a separate first polarizer 408 is not required. In such an embodiment, second polarizer 410 has a plane of polarization oriented 90° relative to the plane of polarization of polarized light source 208.

Collimating lens 402 is preferably a collimating lens or condenser, for collimating light source 208. Collimating lens 402 may be placed in light path 218 on either side of first polarizer 408. The optical and physical characteristics of collimating lens 402 depend on the type of light source being used and the type of image to be eventually projected onto the image detector 212. The optical characteristics of a collimating lens include its focal length, numerical aperture, and F-number (F/#). The physical characteristics of a collimating lens include its material type (glass, plastic, etc.) and shape. Suitable parameters will be apparent to one skilled in the art based on the present description.

A beam splitter 406 is used to form a light path 218 between light source 208 and the illuminated subject 202, and to form a reflected light path 220. The reflected image travels along reflected light path 220 to image detector 212, which detects the reflected image.

As shown in FIG. 4, objective lens 404 is placed in light path 218 and reflected light path 220. Objective lens 404 is preferably an objective lens used to magnify the reflected image. Objective lens 404 is placed co-axially in light path 218 and reflected light path 220. Image detector 212 is located in a magnified image plane of this objective lens. Preferably the objective lens is selected with the lowest magnification level required to visualize the illuminated subject 202.

In a preferred embodiment, image detector 212 is a CCD camera. Suitable devices for image detector 212 include those capable of capturing a high resolution image as defined above. A preferred CCD camera is a Hamamatsu C2400-77 high resolution interline CCD camera. Typically, every one sixtieth of a second an interline CCD camera gathers a field on alternating optical source or wavelength channels. The CCD frames are interlaced, meaning that in normal usage a complete image consists of the combination of an odd and an even field. During the first one-sixtieth of a second, the odd field detects image data only on the odd horizontal lines of the CCD. Then, during the next one-sixtieth of a second, the even field detects image data only on the even horizontal lines of the CCD. When displayed together, the odd and even fields make up a complete image frame. The present invention, however, uses the odd fields and even fields to separately detect image data corresponding to the two light source wavelengths. In the preferred embodiment of the present invention, each odd field detects image data corresponding to the first wavelength of reflected light. Each even field detects image data corresponding to the second wavelength of reflected light. The synchronization signals which gate the integration of the separate fields of the camera are used to switch the light source 208 between the two wavelengths. As a result, two separate images corresponding to the two source wavelengths are detected by a single interline CCD camera. If desired, pixel averaging can be used to fill in the missing lines for each field. The sequence driver 222 synchronizes the detection of the separate odd and even fields by image detector 212 with the light source 208.

Image detector 212 is coupled to video sync separator 214 through composite video signal 226. Video sync separator 214 takes composite video signal 226, and derives its signal components. These signal components are needed by sequence driver 222 to synchronize illumination of the light source 208 with the corresponding optical fields or frames of image detector 212. The signal components of composite video signal 226 include composite sync signal, vertical sync signal, field ID, back porch, and horizontal sync signal. The signal components required by sequence driver 222 are passed in separated video signal 228. Such video sync separation devices are commonly available. A preferred video sync separator 214 is the Colorado Video Model 315TE Sync Separator.

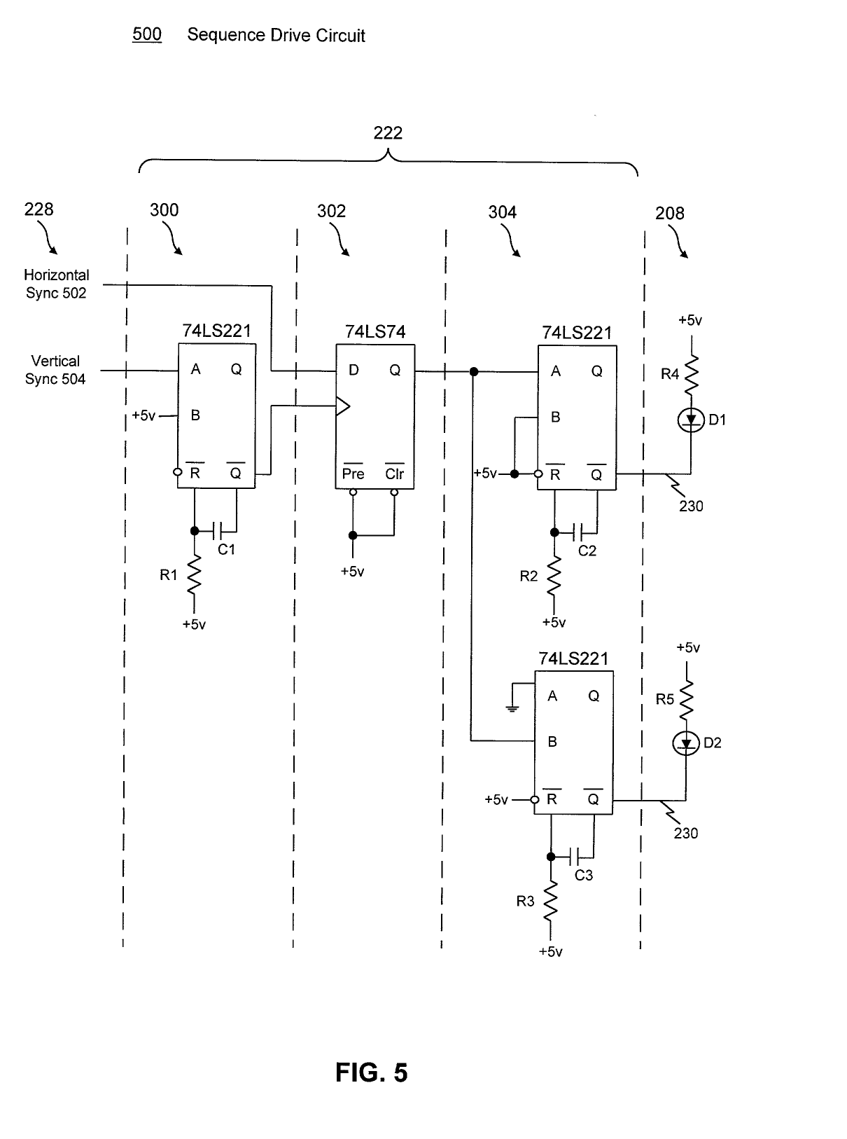

FIG. 5 shows further detail of sequence driver 222 in a circuit diagram describing sequence drive circuit 500, according to a preferred embodiment of the present invention. In the preferred embodiment, video sync separator 214 derives horizontal sync signal 502 and vertical sync signal 504 from composite video signal 226, passing these signals to sequence driver 222 as separated video signal 228. Framing circuit 300 uses vertical sync signal 502 to determine the start of an image detector frame. Synchronization circuit 302 uses the output of framing circuit 300, and horizontal sync signal 504, to determine whether the current image detector field is an even field or an odd field.

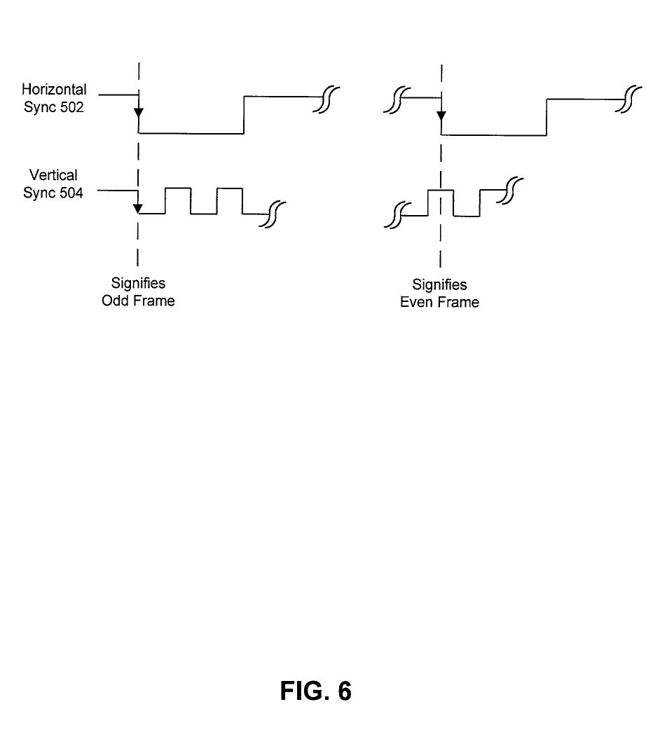

FIG. 6 shows the relationship between the vertical sync signal 502 and horizontal sync signal 504, used to determine whether the current field is even or odd. When both vertical sync signal 502 and horizontal sync signal 504 are undergoing a downward transition, the current field is odd. When vertical sync signal 502 is on a downward transition, and horizontal sync signal 504 is a logical “high”, the current field is even. Whether the field is even or odd determines which LED to trigger. Referring back to FIG. 5, trigger circuit 304 uses the output of synchronization circuit 302 to switch between illumination of the odd LED and the even LED of light source 208. Trigger circuit 304 outputs trigger signal 230, used to trigger the LEDs. In this embodiment, the values for the resistors and capacitors of sequence drive circuit 500 are:

| Component Ref. | Value | |
|---|---|---|
| R1 | 1.5 | kΩ |
| R2, R3 | 20 | kΩ |
| R4, R5 | 100 | Ω |
| C1 | .001 | μF |
| C2, C3 | 1 | μF |

In the preferred embodiment, light source 208 consists of the two LEDs of different wavelengths. Sequence drive circuit 500 controls the triggering of the LEDs, such that each LED is powered in synchronization with the corresponding odd or even optical field of image detector 212.

Image detector 212 is coupled to analyzer 216. Analyzer 216 can be a computer or other type of processing system. A detected image signal 224 representing the reflected image detected by image detector 212 is sent by image detector 212 and received by analyzer 216. Analyzer 216 carries out the processing and analysis of the reflected images received. Analyzer 216 can be configured to carry out these steps through hardware, software, or a combination of hardware and software. In a preferred embodiment, detected image signal 224 passed to analyzer 216 is a composite video signal, hence, detected image signal 224 and composite video signal 226 are the same signal.

Alternate Preferred Embodiment

It will be apparent to one skilled in the art that the preferred embodiment shown in FIG. 4 could be modified to use a broadband light source 208 and a spectral selector 232, located as shown in FIG. 2, instead of monochromatic light sources. Spectral selector 232 would be placed in reflected light path 220 in front of image detector 212. In this alternate preferred embodiment, trigger signal 230 would be used to synchronize spectral selector 232 such that each wavelength passed by spectral selector 232 is synchronized with a corresponding field or frame of image detector 212. As such, sequence driver 222 would not be coupled to light source 208. It will be apparent to one skilled in the art how to modify sequence driver 222 to create a suitable trigger signal 230 for spectral selector 232.

Conclusion

While various embodiments of the present invention have been described above, it should be understood that they have been presented by way of example only, and not limitation. It will be apparent to persons skilled in the relevant art that various changes in form and detail can be made therein without departing from the spirit and scope of the invention. For example, the single detector imaging technique of the present invention can be used in any analytical, in vivo, in vitro, or in situ application that requires optically measuring or visually observing the transmitted or reflected characteristics of an object. Thus, the breadth and scope of the present invention should not be limited by any of the above-described exemplary embodiments, but should be defined only in accordance with the following claims and their equivalents.

What is claimed is:

1. A method for performing bi-chromatic reflected imaging spectrophotometry using a single detector with interline capability, comprising the steps of:
illuminating a subject alternately with a first wavelength of light and a second wavelength of light from a light source, wherein said illumination of said subject by said first wavelength and said second wavelength of light is synchronized with the alternate fields or frames of said single detector, wherein the single detector signals used to integrate said alternate fields or frames of said single detector are provided as synchronization signals to said light source; and
detecting said first wavelength and said second wavelength of light reflected from the subject, respectively, with said alternate fields or frames of said single detector, wherein said detected light is used to perform bi-chromatic imaging spectrophotometry.

2. The method of claim 1, wherein said single detector comprises an interline CCD camera, wherein said detecting step further comprises:
detecting said first wavelength and said second wavelength of light reflected from the subject, respectively, with alternate fields of said interline CCD camera.

3. The method of claim 2, wherein said illuminating step comprises the steps of:
separating vertical sync and horizontal sync signals from a composite video signal received from said single detector;
sequencing said light source using said separated vertical sync and horizontal sync signals such that said first and second wavelengths, respectively, are triggered in synchronization with the odd and even fields of said single detector; and
illuminating said subject with said first wavelength and second wavelength of said light source.

4. The method according to claim 3, wherein said sequencing step comprises the steps of:
framing said first wavelength and said second wavelength with the received vertical sync signal;
synchronizing said first wavelength and said second wavelength with the received horizontal sync signal; and
triggering said light source such that said first wavelength and said second wavelength of light are sequenced in synchronization with the odd and even fields of said single detector.

5. The method according to claim 4, wherein said light source comprises first and second light emitting diodes, wherein said triggering step comprises:
triggering said light emitting diodes, wherein the first light emitting diode emits said first wavelength of light in synchronization with said odd field of said single detector, and wherein the second light emitting diode emits said second wavelength of light in synchronization with said even field of said single detector.

6. The method according to claim 4, wherein said light source comprises first and second laser diodes, wherein said triggering step comprises:
triggering said laser diodes, wherein the first laser diode emits said first wavelength of light in synchronization with said odd field of said single detector, and wherein the second laser diode emits said second wavelength of light in synchronization with said even field of said single detector.

7. The method according to claim 4, wherein said light source comprises an incoherent light source and a spectral selector configured to pass said first wavelength and said second wavelength, wherein said triggering step comprises:
triggering said spectral selector, wherein said spectral selector filters said incoherent light source to pass said first wavelength of light in synchronization with said odd field of said single detector, and wherein said spectral selector filters said incoherent light source to pass said second wavelength of light in synchronization with said even field of said single detector.

8. The method according to claim 7, wherein said spectral selector is a spinning chopper wheel, and wherein triggering said spectral selector step comprises:
triggering said spinning chopper wheel to filter said incoherent light source to pass said first wavelength of light in synchronization with said odd field of said single detector, and to filter said incoherent light source to pass said second wavelength of light in synchronization with said even field of said single detector.

9. The method according to claim 7, wherein said spectral selector is a liquid crystal bandpass filter, and wherein triggering said spectral selector step comprises:
triggering said liquid crystal bandpass filter, to filter said incoherent light source to pass said first wavelength of light in synchronization with said odd field of said single detector, and to filter said incoherent light source to pass said second wavelength of light in synchronization with said even field of said single detector.

10. The method according to claim 7, wherein said spectral selector is an acousto-optic tunable filter, and wherein triggering said spectral selector step comprises:
triggering said acousto-optic tunable filter, to filter said incoherent light source to pass said first wavelength of light in synchronization with said odd field of said single detector, and to filter said incoherent light source to pass said second wavelength of light in synchronization with said even field of said single detector.

11. A method for performing multi-chromatic reflected imaging spectrophotometry using a single detector with multiple field or frame capability, comprising the steps of:
illuminating a subject sequentially with a plurality of light wavelengths from a light source, wherein said illumination of said subject by said plurality of light wavelengths is synchronized with the sequential fields or frames of said single detector, wherein the single detector signals used to integrate said sequential fields or frames of said single detector are provided as synchronization signals to said light source; and
detecting said plurality of light wavelengths reflected from the subject, respectively, with said sequential fields or frames of said single detector, wherein said detected light is used to perform multi-chromatic imaging spectrophotometry.

12. A method for performing bi-chromatic transmitted imaging spectrophotometry using a single detector with interline capability, comprising the steps of:
illuminating a subject alternately with a first wavelength of light and a second wavelength of light from a light source, wherein said illumination of said subject by said first wavelength and said second wavelength of light is synchronized with the alternate fields or frames of said single detector, wherein the single detector signals used to integrate said alternate fields or frames of said single detector are provided as synchronization signals to said light source; and
detecting said first wavelength and said second wavelength of light transmitted through the subject, respectively, with said alternate fields or frames of said single detector, wherein said detected light is used to perform bi-chromatic imaging spectrophotometry.

13. A method for performing multi-chromatic transmitted imaging spectrophotometry using a single detector with multiple field or frame capability, comprising the steps of:
illuminating a subject sequentially with a plurality of light wavelengths from a light source, wherein said illumination of said subject by said plurality of light wavelengths is synchronized with the sequential fields or frames of said single detector, wherein the single detector signals used to integrate said sequential fields or frames of said single detector are provided as synchronization signals to said light source; and
detecting said plurality of light wavelengths transmitted through the subject, respectively, with said sequential fields or frames of said single detector, wherein said detected light is used to perform multi-chromatic imaging spectrophotometry.

14. An apparatus for performing bi-chromatic reflected imaging spectrophotometry using a single detector with interline capability, comprising:
an illuminating region for illuminating a subject alternately with a first wavelength of light and a second wavelength of light from a light source, wherein said illumination of said subject by said first wavelength and said second wavelength of light is synchronized with the alternate fields or frames of said single detector, wherein the single detector signals used to integrate said alternate fields or frames of said single detector are provided as synchronization signals to said light source; and
a detecting region for detecting said first wavelength and said second wavelength of light reflected from the subject, respectively, with said alternate fields or frames of said single detector, wherein said detected light is used to perform bi-chromatic imaging spectrophotometry.

15. The apparatus of claim 14, wherein said single detector comprises an interline CCD camera.

16. The apparatus of claim 15, wherein said illuminating region comprises:
a sync separator for separating vertical sync and horizontal sync signals from a composite video signal received from said single detector;
a sequence driver for sequencing said light source using said separated vertical sync and horizontal sync signals such that said first and second wavelengths, respectively, are triggered in synchronization with the odd and even fields of said single detector; and
a light source for illuminating said subject with said first wavelength and second wavelength of said light source.

17. The apparatus according to claim 16, wherein said sequence driver comprises:
a framing circuit for framing said first wavelength and said second wavelength with the received vertical sync signal;
a synchronization circuit for synchronizing said first wavelength and said second wavelength with the received horizontal sync signal; and
a trigger circuit for triggering said light source such that said first wavelength and said second wavelength of light are sequenced in synchronization with the odd and even fields of said single detector.

18. The apparatus according to claim 17, wherein said light source includes two light emitting diodes.

19. The apparatus according to claim 17, wherein said light source includes two laser diodes.

20. The apparatus according to claim 17, wherein said light source includes an incoherent light source and a spectral selector configured to pass said first wavelength and said second wavelength.

21. The apparatus according to claim 20, wherein said spectral selector is a spinning chopper wheel.

22. The apparatus according to claim 20, wherein said spectral selector is a liquid crystal bandpass filter.

23. The apparatus according to claim 20, wherein said spectral selector is an acousto-optic tunable filter.

24. An apparatus for performing multi-chromatic reflected imaging spectrophotometry using a single detector with multiple field or frame capability, comprising:
an illuminating region for illuminating a subject sequentially with a plurality of light wavelengths from a light source, wherein said illumination of said subject by said plurality of light wavelengths is synchronized with the sequential fields or frames of said single detector, wherein the single detector signals used to integrate said sequential fields or frames of said single detector are provided as synchronization signals to said light source; and
a detecting region for detecting said plurality of light wavelengths reflected from the subject, respectively, with said sequential fields or frames of said single detector, wherein said detected light is used to perform multi-chromatic imaging spectrophotometry.

25. An apparatus for performing bi-chromatic transmitted imaging spectrophotometry using a single detector with interline capability, comprising:
an illuminating region for illuminating a subject alternately with a first wavelength of light and a second wavelength of light from a light source, wherein said illumination of said subject by said first wavelength and said second wavelength of light is synchronized with the alternate fields or frames of said single detector, wherein the single detector signals used to integrate said alternate fields or frames of said single detector are provided as synchronization signals to said light source; and
a detecting region for detecting said first wavelength and said second wavelength of light transmitted through the subject, respectively, with said alternate fields or frames of said single detector, wherein said detected light is used to perform bi-chromatic imaging spectrophotometry.

26. An apparatus for performing multi-chromatic transmitted imaging spectrophotometry using a single detector with multiple field or frame capability, comprising:
an illuminating region for illuminating a subject sequentially with a plurality of light wavelengths from a light source, wherein said illumination of said subject by said plurality of light wavelengths is synchronized with the sequential fields or frames of said single detector, wherein the single detector signals used to integrate said sequential fields or frames of said single detector are provided as synchronization signals to said light source; and
a detecting region for detecting said plurality of light wavelengths transmitted through the subject, respectively, with said sequential fields or frames of said single detector, wherein said detected light is used to perform multi-chromatic imaging spectrophotometry.
